(19) United States
(12) Patent Application Publication
(10) Pub. No.: US 2025/0073410 A1
(43) Pub. Date: Mar. 6, 2025

(54) SYSTEM FOR PULSE CYCLE HARMONIZED VENTILATION AND THE METHOD THEREOF

(71) Applicant: Vikas Khurana, South Abington Township, PA (US)

(72) Inventor: Vikas Khurana, South Abington Township, PA (US)

(21) Appl. No.: 18/286,233

(22) PCT Filed: Apr. 11, 2022
(86) PCT No.: PCT/US2022/024208

§ 371 Date: Oct. 10, 2023

(30) Foreign Application Priority Data
Apr. 11, 2021 (US) 63201076

(51) Int. Cl. A61M 16/00

(52) U.S. Cl. CPC A61M 16/024; A61M 16/0003

(57) ABSTRACT

Disclosed herein is a ventilator system and method, for alveolar micro-circulation enhancement using pulse cycle harmonized ventilation pressure modulation of a patient. The system includes a sensor unit, a controller unit and a supply unit. The sensor unit is configured to sense a set of physiological parameters to determine one or more cardiovascular activities of the patient. The controller unit is communicatively coupled to the sensor unit, and is configured to determine a dynamic pattern of an oxygenated air supply for the patient based on the sensed cardiovascular activity. The supply unit is communicatively coupled to the controller unit and is configured to supply the determined pressure wave to create a dynamic alteration in the air flow pattern of the ventilated air to the patient in response to the cardiovascular activity. Some embodiments of the system also represent the system as an air flow controlling apparatus, capable be coupled to any ventilator system internally or externally to control the flow of air in the dynamic pattern.

### FIELD OF INVENTION

The present disclosure generally relates to a medical device to provide mechanical ventilation to a patient. Specifically, the present disclosure relates to a device for alveolar microcirculation enhancement using pulse cycle harmonized ventilation pressure modulation and the method thereof.

### BACKGROUND

Function of the lungs in the respiratory system is to extract oxygen from the atmosphere and transfer it into the bloodstream, and to release carbon dioxide from the bloodstream into the atmosphere, in a process of gas exchange. Gas exchange in the lungs occurs through millions of small air sacs called alveoli.

Acute respiratory distress syndrome (ARDS) occurs when a fluid builds up in the alveoli of a person. ARDS associated fluid accumulation and inflammation in the alveoli affects the blood oxygen exchange. This leads to hypoxia which deprives various organs in the body system of the oxygen, which in turn affects their functionality.

For treatment of such severe lung complications, the conventional practices usually involve mechanical ventilation together with treatments directed at the underlying cause. Most commonly, a mechanical ventilator provides an external positive end expiratory pressure (PEEP), is used to mitigate end-expiratory alveolar collapse. However, alveolocapillary blood flow decreases with continuous airway pressure in the ventilated patients as continuous pressure collapses the collecting system and does not allow it to fill, leading to a mismatch between the ventilation and perfusion. Further, a prolonged use of ventilator with high PEEP contribute to acute lung injury (ALI) affecting the patient's morbidity and mortality. Thus a trade-off exists between increasing the PEEP to push the fluid out of alveoli and lung injury which occurs because of the increased PEEP, limiting the PEEP that can be safely given.

There are several mechanical ventilators known and conventionally being used as life-saving interventions.

U.S. Pat. No. 8,655,446B2 relates to respiratory therapy control based on cardiac cycle; wherein it relates to a method for delivering airway pressure to a patient, by determining cardiac cycle phase; and controlling the airway pressure based on the cardiac cycle phase, wherein controlling the airway pressure is performed at least in part implantable, which means that the control unit is configured to control airway pressure based on the cardiac cycle phase and the control unit includes at least one implantable component. One embodiment of the invention disclosed in U.S. Pat. No. 8,655,446B2 involves a method for delivering airway pressure to a patient. The method includes determining the cardiac cycle phase and controlling the airway pressure based on the cardiac cycle phase. A parameter indicative of cardiac cycle is sensed and the respiratory pressure therapy is adjusted based on cardiac cycle phase. Modulation of respiratory pressure therapy reinforces the pumping action of the heart and results in improvement in cardiac output with decreased expenditure of myocardial energy output.

Chinese patent application No. CN1541628A discloses a thermotherapeutic cancer treating equipment that consists of high temperature and high-pressure oxygen bed, microwave treating unit, monitor, oxygen supplying unit, hot water supplying unit, outer breathing and beverage replenishing unit.

Chinese patent art CN102441211A relates to a high-intensity pulsating negative pressure therapy device of respiratory disease, including power supply and control circuit; characterized in that: further comprising: a solenoid, a magnet, a multi-plenum vacuum pump A, a multi-plenum vacuum pump B, the intake, outlet, and gas tank shell; wherein the body structure is a multi-linkage solenoid while the air pump A vacuum plenum chamber and a plurality of negative-pressure air pump B, A multi-pump vacuum plenum chamber and a plurality of negative-pressure air pump B can work independently, multi-plenum air pump A of the negative pressure outlet port connected to the intake port a and B multi-tube air chamber B by the negative pressure pump in series to link up to form more than two plenum vacuum pump, entering A after the initial air chamber through the negative pressure suction pump over two plenum, the air intake negative pressure value a times increase, while the noise cancel each other out resulting in more than two air chambers and a vacuum pump connected in series in the pipe, reduce the mechanical movement noise.

US20110142837A1 relates to methods for treating and/or alleviating acute respiratory distress syndrome in an individual diagnosed with or at risk of developing acute respiratory distress syndrome are disclosed. The methods comprise administering a therapeutically effective amount of a complement inhibitor or a TNF-alpha inhibitor to the individual, wherein the complement inhibitor or the TNF-alpha inhibitor reduces or prevents tissue factor production in alveolar neutrophils, thereby treating the ARDS, or delaying or preventing onset of ARDS.

Another U.S. Pat. No. 5,003,976 describes Cardiac and pulmonary physiological analysis via intracardiac measurements with a single sensor; wherein it affords a single sensor within the heart operable to measure a single intracardiac functional parameter and means to derive from that measurement both pulmonary activity and cardiac activity. Both intracardiac pressure fluctuations correlating with the patient's breathing and physiological signals coming from the heart itself can be detected using only one measuring element located within the heart and detecting only one integral intracardiac functional parameter.

Patent application No. RU2282465C2 relates to a device for influencing the cardiovascular system containing a source of compressed air, which is connected to an occlusive inflatable cuff through a receiver with a pressure limiter and gas distribution devices, which can be attached to the patient's body, equipped with pressure sensors, ECG, which together with a pulse oximeter and a blood pressure sensor are connected to the unit for measuring the parameters of the cardiovascular system associated with the control and display unit, characterized in that each gas distribution device contains an occlusion pressure regulator and its relief valve pneumatically connected to the corresponding inflatable cuff, and electrically through a threshold device with a control and indication unit and a cuff pressure sensor, while the control and indication unit is capable of separately controlling the value and the time of supplying pressure to each inflatable cuff, and the bore of the occlusion pressure relief valve in the cuff is selected from the condition for the occlusion pressure relief in man etc. for a time not exceeding the duration of the period of the end of diastole—the beginning of systole of the cardiac cycle. However, patent application No. RU2282465C2 aims towards enhancing the coronary circulation and does not address fixing the alveolar damage through ARDS.

The ventilator systems proposed in the prior art applications carry potential complications such as pneumothorax, airway injury, alveolar damage, ventilator-associated pneumonia and ventilator-associated tracheobronchitis, diaphragm atrophy, decreased cardiac output, and oxygen toxicity. One of the primary complications present in patients mechanically ventilated for long time is acute lung injury (ALI) or prolonged ARDS.

Thus, an advanced ventilator system is required that could overcome the aforementioned problems and must be effective in avoiding damage, injuries, and other complications to the lungs.

### SUMMARY

In view of the foregoing, embodiments of the present disclosure provide a ventilator, a ventilator system (hereinafter interchangeably referred to as “system”) and a method, for alveolar micro-circulation enhancement using pulse cycle harmonized ventilation pressure modulation of a patient. The system includes a sensor unit, a controller unit and a supply unit. The sensor unit is configured to sense a set of physiological parameters to determine one or more cardiovascular activities of the patient. The controller unit that is communicatively coupled to the sensor unit is configured to determine a dynamic pattern of an oxygenated air supply for the patient based on the cardiovascular activities of a patient. The supply unit that is communicatively coupled to the controller unit is configured to supply a determined pressure wave to create the dynamic pattern of the oxygenated air supply to the patient in response to the cardiovascular activity.

The controller unit is configured to determine the dynamic pattern of an oxygenated air supply to the patient by obtaining a variable positive end expiratory pressure (PEEP) value for an external ventilation by synchronizing a ventilation cycle of the ventilator system with a cardiac cycle of the patient. Further, to determine the dynamic pattern, the controller is configured to retain the synchronized ventilation cycle corresponding to early systolic and the entire diastolic phases of the patient's cardiac cycle of the patient. Furthermore, to determine the dynamic pattern, the controller is configured to obtain feedback about ventilation status from the patient and based on the feedback, obtaining one or more specific time intervals.

In an aspect of the present disclosure, the dynamic pattern includes a pre-determined volume of controlled-pressure oxygenated air delivered at specific time intervals, determined by the controller unit, by comparing the set of physiological parameters with a set of corresponding predetermined parameter values.

In some aspects, the supply unit includes an actuator unit having a number of actuators, such that the actuator unit is configured to provide, by way of the number of actuators, the determined pressure variation in the oxygenated air supply (hereinafter interchangeably referred to as “oxygenated air flow”) to the patient instantaneously in the determined dynamic pattern. In another aspect, the actuator unit by way of the number of actuators, based on the calibrated set of internal parameters of the number of actuators, is further configured to provide, the pressure variation of the oxygenated air flow in the dynamic pattern.

In some aspects, upon generation of the dynamic pattern, the controller unit is configured to generate a calibration signal, that is configured to calibrate a set of internal parameters associated with the number of actuators.

In another aspect, the system is configured as an external and/or internal attachment connected externally to a ventilator and is configured to generate oxygenated air in the dynamic pattern

### BRIEF DESCRIPTION OF DRAWINGS

The drawing/s mentioned herein disclose exemplary embodiments of the present invention. Other objects, features, and advantages of the embodiment will be apparent from the following description when read with reference to the accompanying drawings. In the drawings, wherein like reference numerals denote corresponding parts throughout the several views:

The diagrams are for illustration only, which thus is not a limitation of the present disclosure, and wherein:

FIG. 1 illustrates a ventilator system (100) for alveolar microcirculation enhancement using pulse cycle harmonized ventilation pressure modulation for controlling the flow of oxygenated air, according to an embodiment herein;

FIG. 2 illustrated a control unit (104) of the ventilator system (100) for controlling the flow of oxygenated air through the ventilator system (100), according to an embodiment herein;

FIG. 3 illustrates a supply unit (106) of the ventilator system (100) for supplying oxygenated air through the ventilator system (100); and

FIG. 4 illustrates a method (200) for alveolar microcirculation enhancement using pulse cycle harmonized ventilation pressure modulation for controlling the flow of oxygenated air, according to an embodiment herein;

FIG. 5 illustrates a setup of the system (100) for alveolar microcirculation enhancement using pulse cycle harmonized ventilation pressure modulation for controlling the flow of oxygenated air, according to an embodiment herein;

FIG. 6 illustrates a graphical representation of a process (600) for pulse cycle harmonized ventilation pressure modulation, according to an embodiment herein;

FIG. 7 illustrates a graphical representation of pulse cycle of the patient under an effect of different pressure modulation schemes, according to an aspect herein.

### DETAILED DESCRIPTION OF THE PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Unless defined otherwise, all technical and scientific terms used herein have the same meaning as those commonly understood by one of ordinary skill in the art to which this invention belongs.

Throughout the prior art, there remains a need of an advanced ventilator system effective in avoiding damage, injuries, and/or other complications to the lungs.

The system (100) is configured for alveolar microcirculation enhancement using pulse cycle harmonized ventilation pressure modulation for controlling the flow of oxygenated air by way of delivering the oxygenated air in a “dynamic pattern”.

The term “dynamic pattern” as used herein the context of the present disclosure refers to a pre-set (herein after interchangeably referred to as determined) volume of oxygenated air supplied through an external ventilator, wherein the pressure of the oxygenated air through the ventilator is synchronized with the cardiac cycle or the pulse cycle of the patient.

Further, in some aspects, adding to the specification of the dynamic pattern, a pulse cycle harmonized pressure modulation for ventilation may be obtained for an early period of cardiac systole and an entire period of diastole of the cardiac cycle by way of the system (100). In some aspects, adding to the specification of the dynamic pattern, pulse cycle harmonized pressure modulation for ventilation may be obtained only for the diastolic phases of the cardiac cycle. In some aspects, the cardiac cycle synchronized pressure modulated ventilation may be obtained only for the systolic phases of the cardiac cycle.

Furthermore, in some aspects, based on the feedback about ventilation status from the patient the one or more specific time to deliver pulse cycle harmonization based oxygenated air confined only to diastolic phases. In some aspects, the feedback may be obtained by way of a plurality of pulmonary pressures obtained from the patient.

For example, the dynamic pattern of a patient may be such that the pressure of ventilator oxygen is synchronized with the patient's pulse cycle and delivering at first, third and eight diastolic phase based on the feedback about ventilation status by the patient.

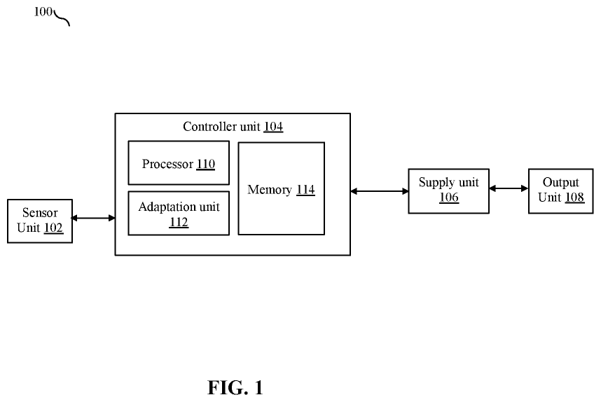

FIG. 1 illustrates a block diagram of a ventilator system (100) (herein after interchangeably referred to as system), according to an aspect of the present disclosure. The system (100) may include a sensor unit (102), a controller unit (104), a supply unit (106), and an output unit (108).

In an embodiment of the present disclosure, the sensor unit (102) may include a plurality of sensors such as blood pressure sensor, breathing sensors or the like and may be configured to sense the set of physiological parameters (hereinafter interchangeably referred to as “set of parameters”) of the patient.

The term “set of parameters” as used herein the context of the present disclosure refers to a set of pulmonary ventilation, pulmonary vasculature and cardiovascular parameters of the patient. The set of parameters may include but not limited to an electrocardiogram (EKG), photoplethysmography (PPG), pulse oximetry, blood pressure value, a rate of inhalation, a depth of breathing, tidal volume, respirate, flow rare, fraction of the inspired oxygen, one or more modes of invasive ventilation including assist-control ventilation (A/C), a synchronized intermittent mandatory ventilation (SIMV), a pressure-control ventilation (PCV), a pressure-support ventilation (PSV), a continuous positive airway pressure (CPAP), a breathing cycle of the patient and non-invasive ventilation including but not limited to a continuous Positive Airway Pressure (CPAP) and a bilevel positive airway pressure (BiPAP). The system (100) may be configured to obtain the pulse cycle and the breathing pattern of the patient by way of one or more of the abovementioned set of parameters.

The non-invasive parameters may further include an electrocardiogramaod oxygen saturation (SPO2), a non-invasive blood pressure (NIBP), a respiration rate and a body temperature. Also, the invasive parameters including a cardiac output, an arterial line pressure, a pulmonary artery wedge pressure, a central venous pressure, a trans-oesophageal echocardiography, a bioimpedance and one or more pulmonary ventilation parameters may be configured to obtain the pulse cycle and the breathing pattern of the patient.

The controller unit (104) may be communicably coupled to the sensor unit (102), and may be configured to compare the set of parameters to a predefined set of parameters. The controller unit (104) may further be configured to analyse the comparison of the set of parameters with the predefined set of parametric values and may be configured to generate a dynamic pattern to supply an oxygenated air flow to the patient. Furthermore, the controller unit (104) may be configured to generate a calibration signal, specifically to deliver to the oxygenated air in the dynamic pattern.

In some aspects of the present disclosure, the supply unit (106) may include a number of actuators (142) to pump and/or deliver a pressurised oxygenated air to the patient in the dynamic pattern determined by the controller unit (104). The controller unit (104) may include a memory (114), configured to store a plurality of data and instructions such as parametric data of state of the number of actuators (142) present in the supply unit (106), configured to deliver the oxygenated air in the dynamic pattern, data of predefined set of parameters for comparison to the set of parameters of the patient, set of the parameters of the patients determined by the sensor unit (102), instruction set of the processor (110) and/or the adaptation unit (112).

The supply unit (106) may be communicatively coupled to the controller unit (104), and configured to supply the determined volume of the oxygenated air to the patient based on the dynamic pattern. The calibration signal generated by the controller unit (104) may be configured to supply the oxygenated air in the dynamic pattern by way of the supply unit (106). The supply unit (106) may be operatively coupled to the output unit (108) and may deliver the oxygenated air to the patient in the dynamic pattern. The output unit (108) may include a mouthpiece, one or more pipes, and/or a band that may be configured to deliver the oxygenated air to the patient. The mouthpiece may be placed inside and/or attached to the oral cavity of the patient. The pipes may be configured to carry the oxygenated air from the supply unit 106 to the mouthpiece. The band may be configured to attach the mouthpiece to the oral cavity and/or the mouth of the patient.

The controller unit (104) may include a processor (110), an adaptive unit (112) and a memory (114). The processor (110) of the controller unit (104) may be configured to compare the set of parameters of the patient to the set of predefined parameters. The processor (110) may further be configured to analyse the comparison and may be configured to generate the dynamic pattern of the oxygenated air supply for the patient. The adaptive unit (112) may be configured to generate a the calibration signal based on the dynamic pattern of the oxygenated air supply determined by the processor (110). Further, the adaptive unit (112) may be communicatively coupled to the supply unit (106) and may configured to communicate the calibration signal to the supply unit (106). The memory (114) of the controller unit (104) may be configured to store a plurality of data including the set of predetermined parameters, plurality of instructions and data, or the like.

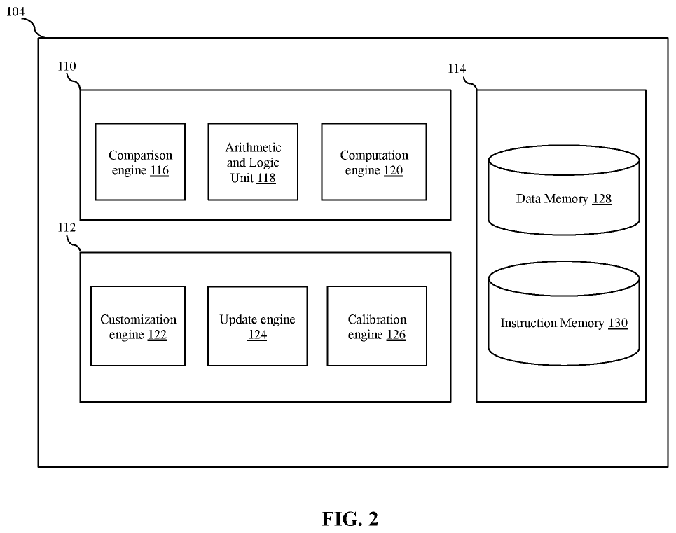

FIG. 2 illustrates a block diagram of the controller unit (104), according to an aspect of the present disclosure. The controller unit (104) may include the processor (110), the adaptation unit (112) and the memory (114). The processor (110) may further include a comparison engine (116), an arithmetic and logical unit (ALU) (118), and a computation engine (120). The comparison engine (116) of the processor (110) may be configured to compare the set of parameters of the patient to the set of predefined parameters stored in the memory (114). Further, the comparison engine (116) may be configured to generate a comparison signal based on the comparison of the set of parameters to the set of predefined parameters. The ALU (118) may be communicatively coupled to the comparison engine (116) and may be configured to perform analysis on the comparison signal. The ALU (118) may further be configured to determine a state of the patient and may be configured to generate a state signal to communicate the state of the patient based on the comparison signal. The computation engine (120) may be communicatively coupled to the ALU (118) and may be configured to determine the dynamic pattern of the oxygenated air supply to the patient. The computation engine (120) may further be configured to generate and/or communicate a dynamic signal to the adaptation unit (112).

In some aspects of the present disclosure, the computation engine (120) may be configured to determine the dynamic pattern of the oxygenated air supply based on a plurality of learning techniques such as artificial intelligence, machine learning and/or deep learning. In another aspect of the present disclosure, the computation engine (120) may be configured to determine the dynamic pattern of the oxygenated air supply based on a plurality of statistical, probabilistic and/or predictive techniques. In another aspect of the present disclosure, the computation engine (120) may be configured to determine the dynamic pattern of the oxygenated air supply based on a plurality of adaptive and/or cognitive mathematical modelling techniques. In another aspect of the present disclosure, the computation engine (120) may be configured to determine the dynamic pattern of the oxygenated air supply based on soft computing techniques such as fuzzy logic. In another aspect of the present disclosure, the computation engine (120) may be configured to determine the dynamic pattern of the oxygenated air supply based on but not limited to a combination of one or more of the learning techniques, the statistical techniques, the probabilistic techniques, the predictive techniques, the adaptive techniques, the cognitive techniques and/or the soft computing techniques.

In some aspects of the present disclosure, the adaptation unit (112) may be configured to generate the calibration signal to calibrate a set of parameters of the supply unit and deliver the oxygenated air in the dynamic pattern determined by the processor (110). The adaptation unit (112) may include a customization engine (122), an update engine (124) and a calibration engine (126).

In some aspects of the present disclosure, the adaptation unit (112) may be communicatively coupled to the supply unit (106). In another aspect of the present disclosure, the adaptation unit (112) may further be coupled with the memory (114) of the controller unit (104). The customization engine (122) may be configured to retrieve the data of state parameters of the set of actuators (142) present in the supply stored in the memory (114) based on the dynamic signal. Further, the customization engine (122) may be configured to calculate changes in the state parameters of plurality of actuators (142), to deliver the supply of the oxygenated air in the dynamic pattern determined. The update engine (124) may be communicatively coupled to the customization engine (122), and may be configured to update the values of state parameters of the number of actuators (122), configured to deliver the oxygenated air in the dynamic pattern. The calibration engine (126) may be communicatively coupled to the update engine (124), and may be configured to generate a the calibration signal to calibrate the state parameters of the number of actuators (142) of the supply unit (106), configured to deliver the oxygenated air in the dynamic pattern to the patient. The memory (114) may be configured to store data and instructions of the system (100) and may include a data memory (128), configured to store the plurality of data of system (100) and an instruction memory (130), configured to store the plurality of instructions of the system (100).

In some aspects of the present disclosure, the memory (114) may be configured as an internal memory of the system 100, examples of which may include a Read-Only Memory (ROM), a Random Access Memory (RAM), a flash memory, a removable storage drive, a hard disk drive (HDD), a solid-state memory, a magnetic storage drive, a Programmable Read Only Memory (PROM), an Erasable PROM (EPROM), and/or an Electrically EPROM (EEPROM), or the like and a combination thereof. In another aspect of the present disclosure, the memory (114) may be configured as an external system memory such as a server, a cloud, a or the like and may be configured to be communicatively coupled with the system (100) by way of wireless and/or wireless communication technologies.

In some aspects of the present disclosure, the sensor unit (102), the controller unit (104), and the supply unit (106) may be communicatively coupled through wired and/or wireless communication technologies such as a Local Area Network (LAN), a Personal Area Network (PAN), a Wireless Local Area Network (WLAN), a Wireless Sensor Network (WSN), Wireless Area Network (WAN), Wireless Wide Area Network (WWAN), a metropolitan area network (MAN), a satellite network, the Internet, a fibre optic network, a coaxial cable network, an infrared (IR) network, a radio frequency (RF) network and a combination thereof.

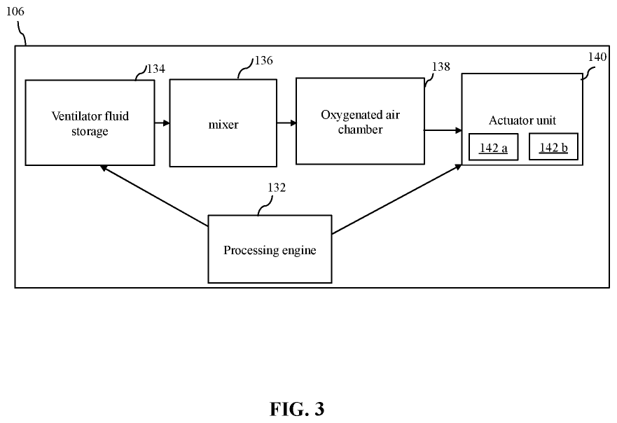

FIG. 3 illustrates a block diagram of the supply unit (106), according to an aspect of the present disclosure. The supply unit (106) may be communicatively coupled to the controller unit (104) and may be configured to receive the calibration signal. The supply unit (106) may include a processing engine (132), a ventilator fluid storage (134), a mixer (136), an oxygenated air chamber (138), and an actuator unit (140). The processing engine (132) may be configured to retrieve the calibration signal and may be configured to generate one or more signals for the generation and actuation of the oxygenated air.

In some aspects of the present disclosure, the processing engine (132) may generate a release signal to release a volume of ventilator fluid from the ventilator fluid storage (134). The ventilator fluid storage (134), may be configured to store a ventilator fluid, not limited to perfluorocarbons (PFCs). Further, the ventilator fluid storage (134) may be coupled to the mixer (136), and upon receipt of the release signal, may be configured to release a volume of the ventilator fluid to the mixer (136). The mixer (136) may be coupled to a non-oxygenated air supply and/or an air chamber and/or an air inlet or the like. Further, the mixer (136), upon receiving the volume of ventilator fluid, may be configured to mix the ventilator fluid with a volume of non-oxygenated air and may be configured to produce a volume of oxygenated air. In another aspect of the present disclosure, the mixer (136) may be configured to include one or more pneumatic circuitry to produce a volume of pressurized oxygenated air. The oxygenated air chamber (138) may be configured to store the volume of oxygenated air produced by the mixer (136). The actuation unit (140) coupled with the oxygenated air chamber (138), may include the number of actuators (142), first through second of the number of actuators (142) are shown as (142a) and (142b) respectively. The actuation unit (140) may further be configured to deliver the oxygenated air in the dynamic pattern to the patient by way of the number of actuators (142). The processing engine (132) may further be configured to generate an actuation signal, to calibrate the state parameters of the number of actuators (142). In some aspects of the present disclosure, the actuator unit (140) may be coupled to the output unit (108), configured to deliver the oxygenated air in the dynamic pattern by way of a mouthpiece.

In another aspect of the present disclosure, the system (100) may be configured as an external air controlling device and/or apparatus (hereinafter interchangeably referred to as “device”) and may be coupled to any state of art ventilator internally or externally to control the flow of air in the dynamic pattern.

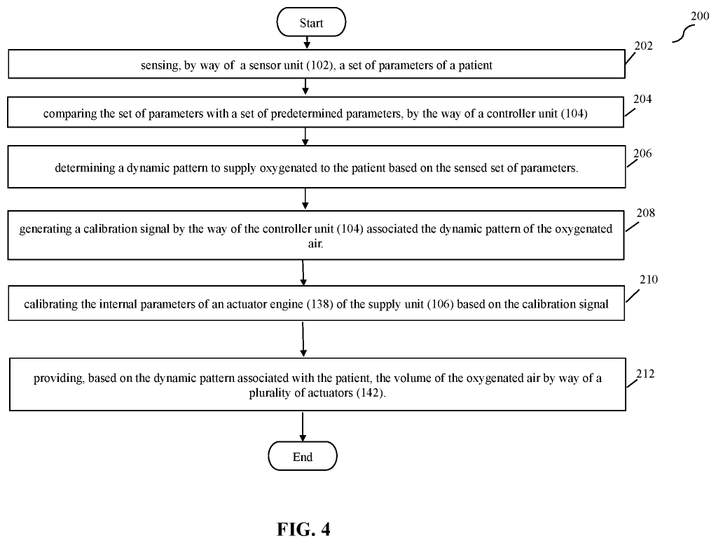

FIG. 4 represents a method (200) that illustrates the functional operation of the system (100) of FIG. 1, according to an aspect of the present disclosure. The method (200) may be considered as an exemplary process to deliver the oxygenated air in the dynamic pattern to the patient performed by the system (100).

At step (202), the system (100) may be configured to select and/or sense the set of parameters of the patient by way of the plurality of sensors such as blood pressure sensor, breathing sensors or the like. The parameters may include but not limited to blood pressure, rate of inhalation, depth of breathing, and/or breathing cycle or the like.

At step (204), the system (100), by way of the controller unit (104), may be configured to obtain the set of parameters and compare the set of parameters to the predefined set of parameters.

At step (206), the system (100), by way of the controller unit (104), may be configured to analyse the comparison of the set of parameters and the set of predefined parameters and may be configured to determine the dynamic pattern of oxygenated air supply to the patient.

At step (208), the system (100), by way of the controller unit (104) may be configured to generate the calibration signal to calibrate the actuators (142) of the supply unit (106).

At step (210), the system (100), by way of the processing unit (132) of the supply unit (106), may be configured to release the volume of the ventilator fluid from the ventilator fluid storage (134) upon receiving the calibration signal. Further, at step (210), the system (100), by way of the mixer (136) of the supply unit (106), may be configured to produce and/or store the volume of oxygenated air in the oxygenated air chamber (138) by mixing the ventilator fluid with the volume of air. In another aspect of the present disclosure, at step (210), the system (100), by way of the pneumatic circuitry of the mixer (136), may be configured to produce pressurized oxygenated air. Furthermore, the system (100), by way of the processor unit (132) of the supply unit (106), may be configured to calibrate the state parameters of the number of actuators (142) of the actuator unit (140).

At step (212), the system (100), by way of the actuator unit (138) of the supply unit (106), may be configured to provide the oxygenated air to the patient in the dynamic pattern determined by the controller unit in step (206).

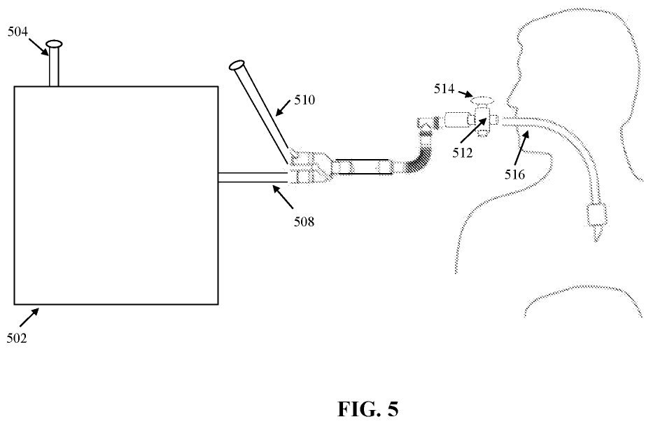

FIG. 5 illustrates a setup of the system (100) for alveolar microcirculation enhancement using pulse cycle harmonized ventilation pressure modulation for controlling the flow of oxygenated air, according to an embodiment herein. The setup may include a ventilator (502), an air inlet tube (504), an inhalation tube (508), an exhalation tube (510), a pressure regulator (512), a controller mechanism (not shown) controlling the pressure regulator and an input pipe (516).

In an aspect, the ventilator (502) may intake air from the surroundings through one or more air inlet tubes (504). Further, the ventilator (502) may include a volume of ventilator fluid for oxygenation of air. The ventilator (502) may further include pneumatic circuitry and/or one or more actuators to deliver oxygenated air. The inhalation tube (508) may be configured to guide the oxygenated air outside the ventilator (502). The exhalation tube (510) may be configured to guide the flow of expiratory air out. The pressure regulator (512) may be configured to control the pressure of the oxygenated air as well as the flow of the oxygenated air in the dynamic pattern to facilitate pulse cycle harmonized ventilation pressure modulation to the oxygenated air. The pressure regulator may include a piston (514) to enable the flow of oxygenated air in the dynamic pattern. The input pipe (516) may be configured as a mouthpiece to deliver the oxygenated air in the dynamic pattern to the patient.

In some aspects, the ventilator (502) for pulse cycle harmonized pressure modulation includes a sensor unit (102) configured to sense a set of physiological parameters, to determine one or more cardiovascular activities of a patient. The ventilator (502) may further include a controller unit (104) that is communicatively coupled to the sensor unit (102), and configured to determine a dynamic pattern of an oxygenated air supply for the patient based on the cardiovascular activities of a patient. Furthermore, the ventilator (502) may include a supply unit (106) that is communicatively coupled to the controller unit (104), and may be configured to supply a determined pressure wave to create the dynamic pattern of the oxygenated air supply to the patient. In some aspect of the present disclosure, the pressure regulator (512) may be placed adjacent to the input pipe, such that the oxygenated air in the dynamic pattern can be pumped directly to the patient with minimal loss in pressure.

In an aspect of the present disclosure, the piston (514) may be used to deliver the medication deeply to the lung. In other aspect, the medication may be delivered to the patient as an aerosol and the piston (514) may be used to deliver the medication through a synchronized manner to a ventilator setting of inhalation.

In some aspects, the pulse cycle harmonized pressure modulated ventilation may be applied to correct a ventilation perfusion mismatch and/or one or more ventilation to perfusion ratio (V/Q) defects. The V/Q defects may lead to a condition, wherein one or more areas of the lung receive oxygen without any blood flow and/or one or more areas of the lungs receive blood flow but do not receive oxygen. In some aspects, the pulse cycle harmonized pressure modulated ventilation may be applied to a bi-level positive airway pressure (BiPAP) machine to correct the ventilation perfusion mismatch and/or one or more ventilation to perfusion ratio (V/Q) defects.

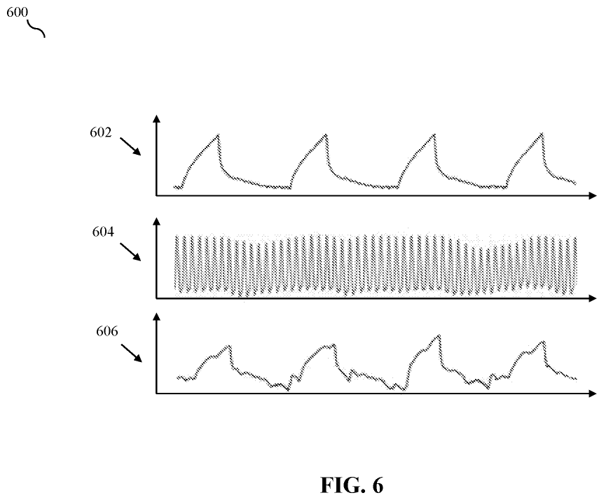

FIG. 6 illustrates a graphical representation of a process (600) for pulse cycle harmonized ventilation pressure modulation, according to an aspect herein. In an aspect of the present disclosure, (602) may represent a pulse representing the pressure of air throw by a ventilator. (604) may represent a pulse of the patient's cardiac cycle. Further, (606) may represent the pulse of ventilator synchronized with the cardiac cycle of the patient, such that the pressure of air delivery by the ventilator is modulated in accordance to the cardiac cycle of the patient, thereby resulting in pulse cycle harmonized ventilation pressure modulation.

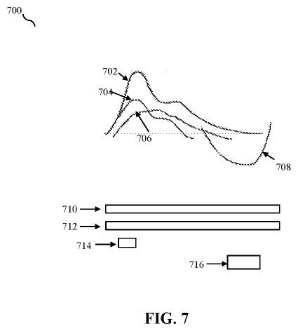

FIG. 7 illustrates a graphical representation of pulse cycle of the patient under an effect of different pressure modulation schemes, according to an aspect herein. In an aspect, (710) may be a representation for a constant Pressure throughout the entire cardiac cycle, (712) may represent a negative pressure PEEP during systole, (714) may represent a positive pulse cycle harmonized pressure modulation (PCHPM) PEEP in the systolic cardiac cycle phase, (716) may represent a second PCHPM PEEP in the diastolic cardiac cycle phase.

In an aspect, (710) may be used as a constant pressure during the entire cardiac cycle and is represented by (706). The pulse represented by (702) may represent the baseline pulse cycle. After (714), a positive pressure during systole as the blood starts flowing into the capillary circulation, the (714) may be applied for a small duration causing blunting of the blood flow peak in the area and may lead to decreased outflow of fluid from a capillary system resulting in (704). The (716) a positive pressure may be applied to during the diastolic phase of the cardiac cycle. With the application of (716), the (702) may be modified to (708) and may lead to increased removal of fluid by a venous system.

In some aspects, the PCHPM may be set up in a plurality of ways. In an aspect, a fixed combinations of PCHPM and FIO₂ settings to reach an oxygenation goal range. Further, PCHPM may be set up in accordance with one or more physiologic variables (i.e maintaining the pulse oximetry more than 95% by adjusting the inspired oxygen values permitted and PCHPM while maintaining a maximum pressure<60 cm H2O). Furthermore, the PCHPM may be set up according to end-expiratory transpulmonary pressure (i.e., difference between airway-opening pressure and pleural pressure, with pleural pressure estimated from oesophageal pressure).

One or more strategies may be used to adjust PCHPM in a way similar to adjusting PEEP in patients with ARDS guided by oesophageal pressure, Stress Index, static airway pressure-volume curve, as described elsewhere and should be known to the person skilled in the art. It must be understood to any person known to the art that FIG. 6 and FIG. 7 may not represent an actual representations obtained through experimentation and may be considered only to explain one or more effects and/or phenomenon due to the system (100).

In some aspects, a combined application of pressure during the systolic phase may prevent the capillary fluid from coming out into the alveoli during the systolic phase while the diastolic positive pressure is pushing the previously leaked fluid back into the vasculature in the diastolic phase of the capillary blood flow.

In some aspects, a selective application of pressure only during the diastolic phase may allow the interstitial fluid to come out during the systolic phase while pushing the excess fluid back in the diastolic phase of the capillary blood flow. Using cardio-synchronized compressions to increase the tissue pressure only during the diastolic phase of blood flow in the capillary, increased fluid may return into the venous system and be removed from the tissue and may decrease the tissue pressure, leading to increase in blood flow and interstitial fluid flow during the arterial phase, providing better nutrients and oxygenation to the tissue.

In some aspects of the present disclosure, the cardiovascular activity may be determined through a plurality of parameters such a plurality of blood vibrations, feedback from an indwelling pressure sensor catheter, an EKG signal, and/or a doppler flow value.

In some aspects, a vibration sensor may be used to sense a start of the movement of the blood. The start of the movement of the blood may not be the actual blood-flow. In some aspects, feedback from an indwelling pressure sensor catheters may be obtained by way of one or more indwelling pressure sensor catheters that may be placed into one or more major vascular structures close to a site of treatment. The indwelling pressure sensor catheter may sense an actual pressure and may provide feedback to guide the adjustment in the cuff pressure.

A heart pumps blood in a classic waveform that is critical to mimic physiological functioning. In some aspects, one or more points on the classic waveform may be iteratively altered and may have one or more effects on the microcirculation of the treatment part. Further, the alterations in the waveform may have one or more physiological effect for an individual patient.

In some aspects, an electrical activity of the heart may be faster than an actual flow of blood and may be detected from different parts of the body including the extremities, chest or a combination of multiple points by way of an EKG signal. In some aspects, the doppler flow value may detect the actual flow of blood and may be used to further modify the pressure.

In some aspect, the PCHPM may enhance the microvascular flow by providing one or more shear forces to a microvasculature. The shear forces to the microvasculature may improve one or more endothelial functions of a body.

In an aspect of the present disclosure, the system (100) may be configured to sense a cardiac activity, predict when the peak of the pulse is going to come into the lungs, and give a positive pressure supplement to the ventilation air flow.

In an aspect, the system (100) may suppress a peak of the pulse wave the lungs with the increased positive pressure which may be responsible for further exudation of fluids into the alveoli of the lung.

In some aspects of the present disclosure, the system (100) may facilitate a patient with alveolar microcirculation enhancement using pulse cycle harmonized ventilation pressure modulation providing effective solution to treat acute respiratory distress syndrome (ARDS) and other complications of the lungs. Further, in some aspect of the present disclosure, the system (100) may be configured to avoid a mismatch between ventilation and perfusion caused by decrease in alveolocapillary blood flow with continuous airway pressure in ventilated patients or positive end expiratory pressure (PEEP).

In some aspect of the present disclosure, the system (100) may be configured to systematically modulate and create pulse harmonization on top of the ventilator pressure to avoid damaging effects of positive end expiratory pressure (PEEP). The system (100) may be configured as assisted ventilation system using an instant mechanical ventilation device to avoid alveolar damages and instantaneous enhanced blood oxidation. Further, the system (100) may be configured to be selectively operated only when it is required. Furthermore, the system (100) may be configured to supply a controlled small respiratory minute volume of oxygen enriched air or respiratory fluid at certain intervals at optimized moments of inhalation in the breathing cycles of the patient with ARDS and/or other complications of lungs.

In some aspects of the present disclosure, the system (100) for alveolar microcirculation enhancement using pulse cycle harmonized ventilation pressure modulation may be configured to ensure optimum safety to the alveolar conditions of the patient with ARDS/lung complications as well as to ensure enhanced blood pressure in the alveolar microcapillaries towards achieving higher rate of oxygenation and better provision of nutrients to the tissues.

Certain terms are used throughout the following description and claims to refer to particular features or components. As one skilled in the art will appreciate, different persons may refer to the same feature or component by different names. This document does not intend to distinguish between components or features that differ in name but not structure or function. While various embodiments of the present disclosure have been illustrated and described, it will be clear that the present disclosure is not limited to these embodiments only. Numerous modifications, changes, variations, substitutions, and equivalents will be apparent to those skilled in the art, without departing from the spirit and scope of the present disclosure, as described in the claims.

We claim:

1. A ventilator system (100) for pulse cycle harmonized pressure modulation comprises:
a) a sensor unit (102) configured to sense a set of physiological parameters, to determine one or more cardiovascular activities of a patient;
b) a controller unit (104) that is communicatively coupled to the sensor unit (102), and is configured to determine a dynamic pattern of an oxygenated air supply for the patient based on the cardiovascular activities of the patient; and
c) a supply unit (106) that is communicatively coupled to the controller unit (104), and configured to supply a determined pressure wave to create the dynamic pattern of the oxygenated air supply to the patient.

2. The ventilator system (100) of claim 1, wherein the controller unit (104) configured to determine the dynamic pattern of the oxygenated air supply by:
a) obtaining a variable positive end expiratory pressure (PEEP) for an external ventilation by synchronizing a ventilation cycle of the ventilator system (100) with a cardiac cycle of the patient;
b) retaining only the synchronized ventilation cycle corresponding to diastolic phases of the patient's cardiac cycle; and
c) obtaining feedback about ventilation status of the patient from the sensor unit (102), and
d) obtaining one or more specific time intervals for supply of oxygenated air based on the feedback obtained at step c).

3. The ventilator system (100) of claim 1, wherein the controller unit (104) is configured to determine an early systolic and an entire diastolic phase of the cardiac cycle of the patient.

4. The ventilator system (100) of claim 1 or 2, wherein the dynamic pattern comprising a pre-set volume of a pressure modulated oxygenated air supply at specific time intervals, determined by the controller unit (104), based on the set of parameters of the patient.

5. The ventilator system (100) of claim 1, wherein to determine the dynamic pattern, the controller unit (104) is configured to compare the set of physiological parameters of the patient with a set of corresponding pre-determined parameter values.

6. The ventilator system (100) of claim 1, the supply unit (106) further comprising an actuator unit (138) having a plurality of actuators (142) such that the actuator unit (138) is configured to provide, by way of the plurality of actuators (142), the determined pressure variation in the oxygenated air supply to the patient instantaneously in the determined dynamic pattern.

7. The ventilator system (100) of claim 1, wherein, upon generation of the dynamic pattern, the controller unit (104) is configured to generate a calibration signal, that is configured to calibrate a set of internal parameters associated with the plurality of actuators (142).

8. The ventilator system (100) of the claim 1, wherein the actuator unit (138) by way of the plurality of actuators (142), based on the calibrated set of internal parameters of the plurality of actuators (142), is further configured to provide, the volume of the oxygenated air in the dynamic pattern.

9. The ventilator system (100) of claim 1, wherein the system is connected externally to a ventilator and is configured to generate oxygenated air in the dynamic pattern.

10. The ventilator system (100) of claim 1 is connected externally to a ventilator and is configured to generate oxygenated air in the dynamic pattern.

11. A method (200) for supplying oxygenated air, the method (200) comprises:
a) sensing, by way of a sensor unit (102), a set of physiological parameters of a patient to determine one or more cardiovascular activities of a patient;
b) comparing the set of physiological parameters with a set of predetermined parameters, by the way of a controller unit (104);
c) determining a dynamic pattern to supply oxygenated air to the patient based on the sensed set of physiological parameters, wherein the dynamic pattern comprises a preset volume of oxygenated air to be provided at corresponding time intervals;
d) generating a calibration signal by the way of the controller unit (104), wherein the calibration signal is associated the dynamic pattern of the oxygenated air;
e) calibrating the internal parameters of an actuator engine (138) of the supply unit (106) based on the calibration signal; and
f) providing, based on the dynamic pattern associated with the patient, the volume of the oxygenated air by way of a plurality of actuators (142).

12. The method of claim 11, wherein determining the dynamic pattern comprising:
a) obtaining a variable positive end expiratory pressure (PEEP) for an external ventilation by synchronizing a ventilation cycle of the ventilator system (100) supplying oxygenated air to the patient with a cardiac cycle of the patient;
b) retaining only the synchronized ventilation cycle corresponding to diastolic phases of the patient's cardiac cycle of the patient;
c) obtaining feedback about ventilation status from the patient and based on the feedback, obtaining one or more specific time intervals.

13. The method of 11 or 12 providing, by way of the plurality of actuators (142), the determined volume of the oxygenated air to the patient instantaneously in the determined dynamic pattern.

14. A ventilator (502) comprises:
a) a sensor unit (102) configured to sense a set of physiological parameters, to determine one or more cardiovascular activities of a patient;
b) a controller unit (104) that is communicatively coupled to the sensor unit (102), and configured to determine a dynamic pattern of an oxygenated air supply for the patient based on the cardiovascular activities of a patient; and
c) supply unit (106) that is communicatively coupled to the controller unit (104), and configured to supply a determined pressure wave to create the dynamic pattern of the oxygenated air supply to the patient.

15. The ventilator (502) of claim 14, wherein the controller unit (104) is configured to determine the dynamic pattern of the oxygenated air supply to the patient by:
a) obtaining a variable positive end expiratory pressure (PEEP) for an external ventilation by synchronizing a ventilation cycle of the ventilator system (100) supplying oxygenated air to the patient with a cardiac cycle of the patient;
b) retaining only the synchronized ventilation cycle corresponding to diastolic phases of the patient's cardiac cycle of the patient; and
c) obtaining feedback about ventilation status of the patient by way of the sensor unit (102), and based on the feedback, obtaining one or more specific time intervals.

16. The ventilator (502) of claim 14, wherein the controller unit (104) is configured to determine an early systolic and an entire diastolic phase of the cardiac cycle of the patient.

17. The ventilator (502) of claim 14 or 15, wherein the dynamic pattern comprising a pre-set volume of a pressure modulated oxygenated air supply at specific time intervals, determined by the controller unit (104), based on the set of physiological parameters of the patient.

18. The ventilator (502) of claim 14, wherein to determine the dynamic pattern, the controller unit (104) is configured to compare the set of physiological parameters with a set of corresponding pre-determined parameter values.

19. The ventilator (502) of claim 14, the supply unit (106) further comprising an actuator unit (138) having a plurality of actuators (142) such that the actuator unit (138) is configured to provide, by way of the plurality of actuators (142), the determined pressure variation in the oxygenated air supply to the patient instantaneously in the determined dynamic pattern.

20. The ventilator (502) of the claim 14, wherein the actuator unit (138) by way of the plurality of actuators (142), based on the calibrated set of internal parameters of the plurality of actuators (142), is further configured to provide, the volume of the oxygenated air in the dynamic pattern.
